(19) United States
(12) Patent Application Publication
(10) Pub. No.: US 2023/0067239 A1
(43) Pub. Date: Mar. 2, 2023

(54) MONITORING AND RESPONSE VIRTUAL ASSISTANT FOR A COMMUNICATION SESSION

(71) Applicant: AT&T Intellectual Property I, L.P., Atlanta, GA (US)

(72) Inventor: Rashmi Palamadai, Naperville, IL (US)

(73) Assignee: AT&T Intellectual Property I, L.P., Atlanta, GA (US)

(21) Appl. No.: 17/459,586

(22) Filed: Aug. 27, 2021

(51) Int. Cl. G08B 13/00; G01D 21/02; A61B 5/0205; A61B 5/021; A61B 5/024; A61B 5/145; A61B 5/08; A61B 3/113

(52) U.S. Cl. CPC G08B 13/00; G01D 21/02; A61B 5/0205; A61B 5/021; A61B 5/024; A61B 5/14542; A61B 5/14546; A61B 5/14532; A61B 5/0816; A61B 3/113; G08B 21/02

(57) ABSTRACT

Aspects of the subject disclosure may include, for example, monitoring a communication session in which a user participates, the user being at a location; obtaining sensor information indicative of a physical environment in a vicinity of the location; determining, based at least in part upon the sensor information, whether a person other than the user is in the physical environment, resulting in a determination; and responsive to the determination being that the person is in the physical environment: categorizing, based at least in part upon the sensor information, the person into one class of a plurality of classes of people; generating an alert, the alert indicating to which class the person has been categorized; and providing the alert via the communication session to a communication device being used by the user. Other embodiments are disclosed.

### FIELD OF THE DISCLOSURE

The subject disclosure relates to a monitoring and response virtual assistant for a communication session.

### BACKGROUND

Various types of communication sessions exist. Communication sessions can include telephone conferences, video conferences and/or extended reality (sometimes referred to herein as XR) communication sessions. XR communication sessions can include augmented reality (sometimes referred to herein as AR), mixed reality (sometimes referred to herein as MR), and/or virtual reality (sometimes referred to herein as VR). In AR, virtual information is overlaid on a real-world view. MR is similar to AR, but in MR digital content (e.g., 3D digital content) can be spatially aware and responsive. In VR, the view is simulated. In various examples, AR communication sessions, MR communication sessions, and/or VR communication sessions can be provided via googles, glasses, or other display screens.

### BRIEF DESCRIPTION OF THE DRAWINGS

Reference will now be made to the accompanying drawings, which are not necessarily drawn to scale, and wherein:

FIG. 1 is a block diagram illustrating an example, non-limiting embodiment of a communication network in accordance with various aspects described herein.

FIG. 2A is a block diagram illustrating an example, non-limiting embodiment of a system (that can function fully or partially within the communication network of FIG. 1) in accordance with various aspects described herein.

FIG. 2B is a block diagram illustrating an example, non-limiting embodiment of a system flow (that can function fully or partially within the communication network of FIG. 1) in accordance with various aspects described herein.

FIG. 2C depicts an illustrative embodiment of a method in accordance with various aspects described herein.

FIG. 2D depicts an illustrative embodiment of a method in accordance with various aspects described herein.

FIG. 2E depicts an illustrative embodiment of a method in accordance with various aspects described herein.

FIG. 2F is a block diagram illustrating an example, non-limiting embodiment of a map in accordance with various aspects described herein.

FIG. 3 is a block diagram illustrating an example, non-limiting embodiment of a virtualized communication network in accordance with various aspects described herein.

FIG. 4 is a block diagram of an example, non-limiting embodiment of a computing environment in accordance with various aspects described herein.

FIG. 5 is a block diagram of an example, non-limiting embodiment of a mobile network platform in accordance with various aspects described herein.

FIG. 6 is a block diagram of an example, non-limiting embodiment of a communication device in accordance with various aspects described herein.

### DETAILED DESCRIPTION

The subject disclosure describes, among other things, illustrative embodiments for monitoring and/or responding during a user's communication session. Other embodiments are described in the subject disclosure.

One or more aspects of the subject disclosure include a virtual assistant (sometimes referred to herein as VA) that performs monitoring and/or responding during a user's communication session. In various examples, the communication session can comprise an XR communication session, a VR communication session, an AR communication session, an MR communication session, or any combination thereof. In various examples, the monitoring can be monitoring of a user's interaction and the responding can be responding to the user's interaction. In various examples, the monitoring can be monitoring of an external event (and/or person) and the responding can be responding to the external event (and/or person).

One or more aspects of the subject disclosure include monitoring and/or responding during a user's XR communication session.

As described herein, there can be a situation where a user is deeply involved within a communication session (e.g., a VR session and/or an AR session). In various examples, mechanisms can be provided to monitor what is happening outside the communication session and, if necessary, alert the user to exit the communication session and/or to take appropriate action.

As described herein, there can be a situation where a user is involved within a communication session (e.g., a VR session and/or an AR session) and the user has a medical/health issue. In various examples, mechanisms can facilitate providing assistance to deal with the issue—e.g., by temporarily handling and/or alerting emergency services.

As described herein, various embodiments can provide mechanisms to enable a user to choose only to be interrupted by specific people and/or for specific reasons. In various examples, any type of interruption could need to be qualified (e.g., based on urgency and/or who is requesting for the user's attention). In various examples, mechanisms can be provided (e.g., while the user is in a VR session and/or AR session) such that a VA can track multiple occurrences of health issues as it is monitoring body functions, and alert/keep track of patterns of issues (e.g., falling asleep, losing balance, losing blood pressure, etc.).

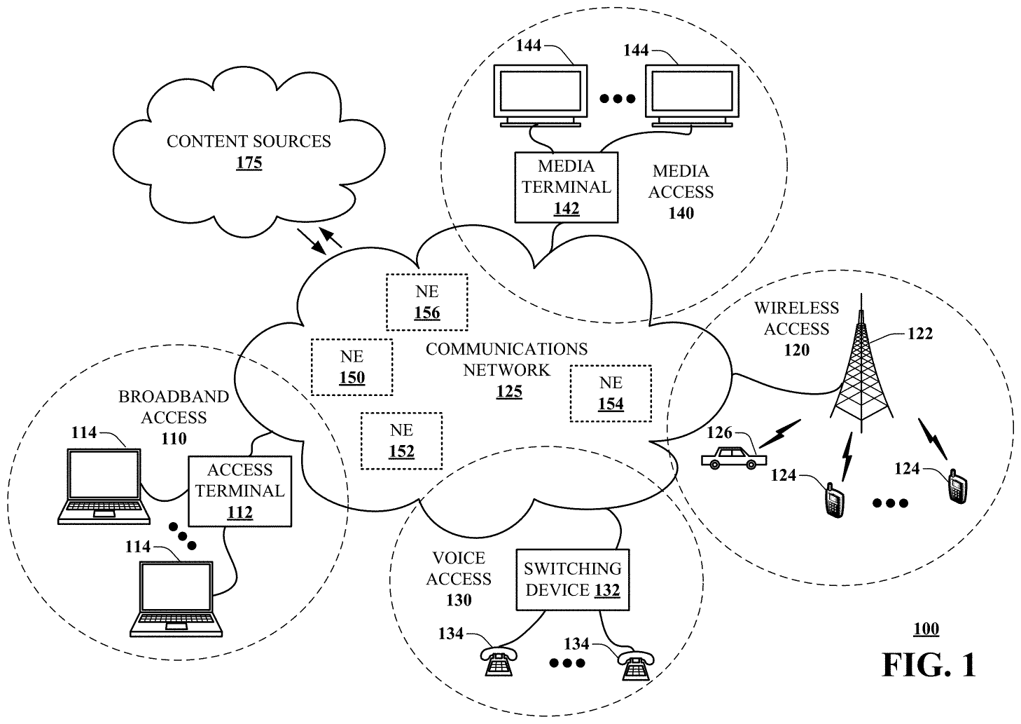

Referring now to FIG. 1, a block diagram is shown illustrating an example, non-limiting embodiment of a system 100 in accordance with various aspects described herein. For example, system 100 can facilitate in whole or in part monitoring and/or responding during a user's communication session. In particular, a communications network 125 is presented for providing broadband access 110 to a plurality of data terminals 114 via access terminal 112, wireless access 120 to a plurality of mobile devices 124 and vehicle 126 via base station or access point 122, voice access 130 to a plurality of telephony devices 134, via switching device 132 and/or media access 140 to a plurality of audio/video display devices 144 via media terminal 142. In addition, communication network 125 is coupled to one or more content sources 175 of audio, video, graphics, text and/or other media. While broadband access 110, wireless access 120, voice access 130 and media access 140 are shown separately, one or more of these forms of access can be combined to provide multiple access services to a single client device (e.g., mobile devices 124 can receive media content via media terminal 142, data terminal 114 can be provided voice access via switching device 132, and so on).

The communications network 125 includes a plurality of network elements (NE) 150, 152, 154, 156, etc. for facilitating the broadband access 110, wireless access 120, voice access 130, media access 140 and/or the distribution of content from content sources 175. The communications network 125 can include a circuit switched or packet switched network, a voice over Internet protocol (VoIP) network, Internet protocol (IP) network, a cable network, a passive or active optical network, a 4G, 5G, or higher generation wireless access network, WIMAX network, UltraWideband network, personal area network or other wireless access network, a broadcast satellite network and/or other communications network.

In various embodiments, the access terminal 112 can include a digital subscriber line access multiplexer (DSLAM), cable modem termination system (CMTS), optical line terminal (OLT) and/or other access terminal. The data terminals 114 can include personal computers, laptop computers, netbook computers, tablets or other computing devices along with digital subscriber line (DSL) modems, data over coax service interface specification (DOCSIS) modems or other cable modems, a wireless modem such as a 4G, 5G, or higher generation modem, an optical modem and/or other access devices.

In various embodiments, the base station or access point 122 can include a 4G, 5G, or higher generation base station, an access point that operates via an 802.11 standard such as 802.11n, 802.11ac or other wireless access terminal. The mobile devices 124 can include mobile phones, e-readers, tablets, phablets, wireless modems, and/or other mobile computing devices.

In various embodiments, the switching device 132 can include a private branch exchange or central office switch, a media services gateway, VoIP gateway or other gateway device and/or other switching device. The telephony devices 134 can include traditional telephones (with or without a terminal adapter), VoIP telephones and/or other telephony devices.

In various embodiments, the media terminal 142 can include a cable head-end or other TV head-end, a satellite receiver, gateway or other media terminal 142. The display devices 144 can include televisions with or without a set top box, personal computers and/or other display devices.

In various embodiments, the content sources 175 include broadcast television and radio sources, video on demand platforms and streaming video and audio services platforms, one or more content data networks, data servers, web servers and other content servers, and/or other sources of media.

In various embodiments, the communications network 125 can include wired, optical and/or wireless links and the network elements 150, 152, 154, 156, etc. can include service switching points, signal transfer points, service control points, network gateways, media distribution hubs, servers, firewalls, routers, edge devices, switches and other network nodes for routing and controlling communications traffic over wired, optical and wireless links as part of the Internet and other public networks as well as one or more private networks, for managing subscriber access, for billing and network management and for supporting other network functions.

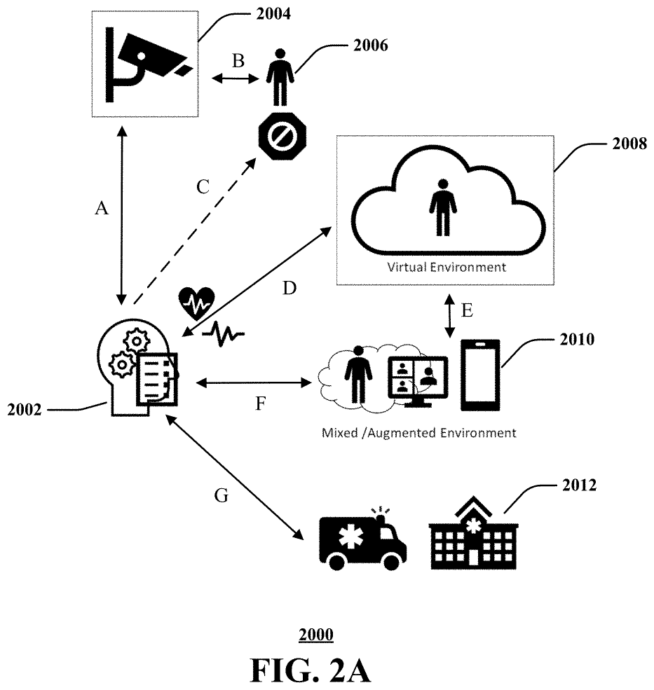

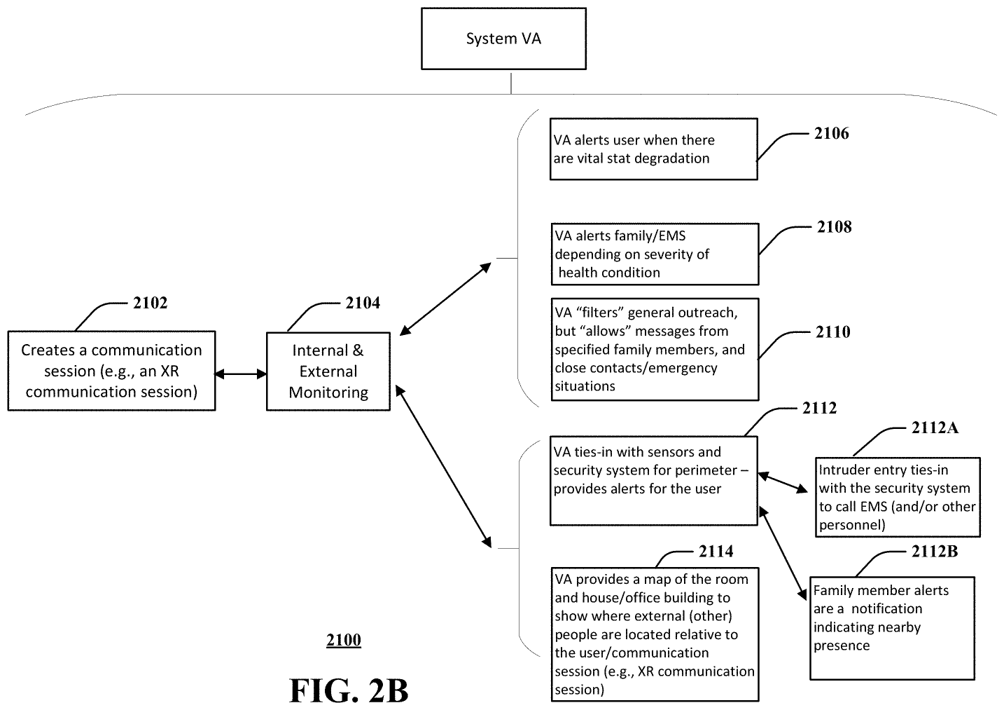

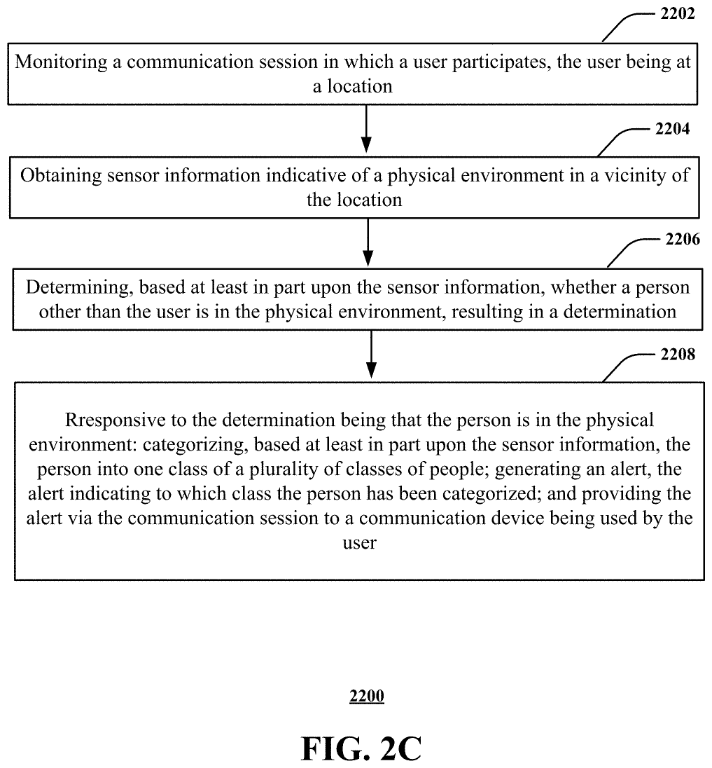

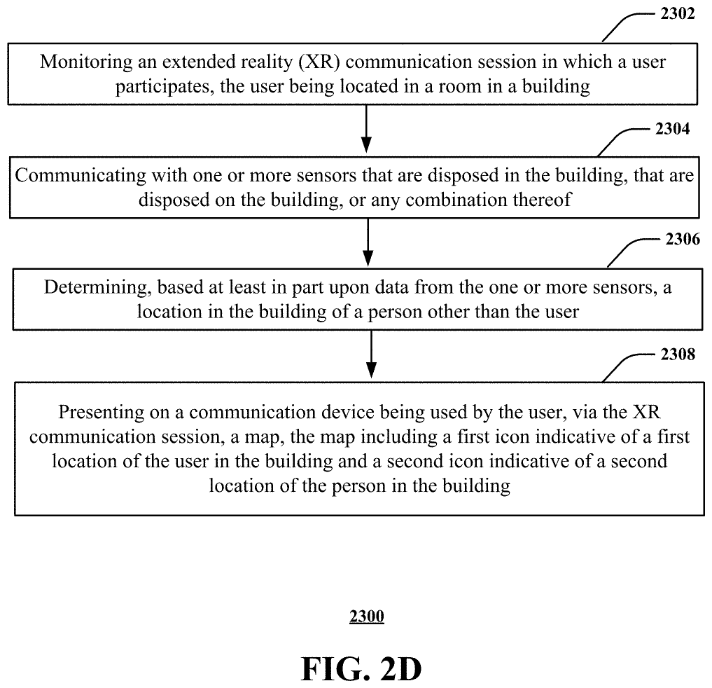

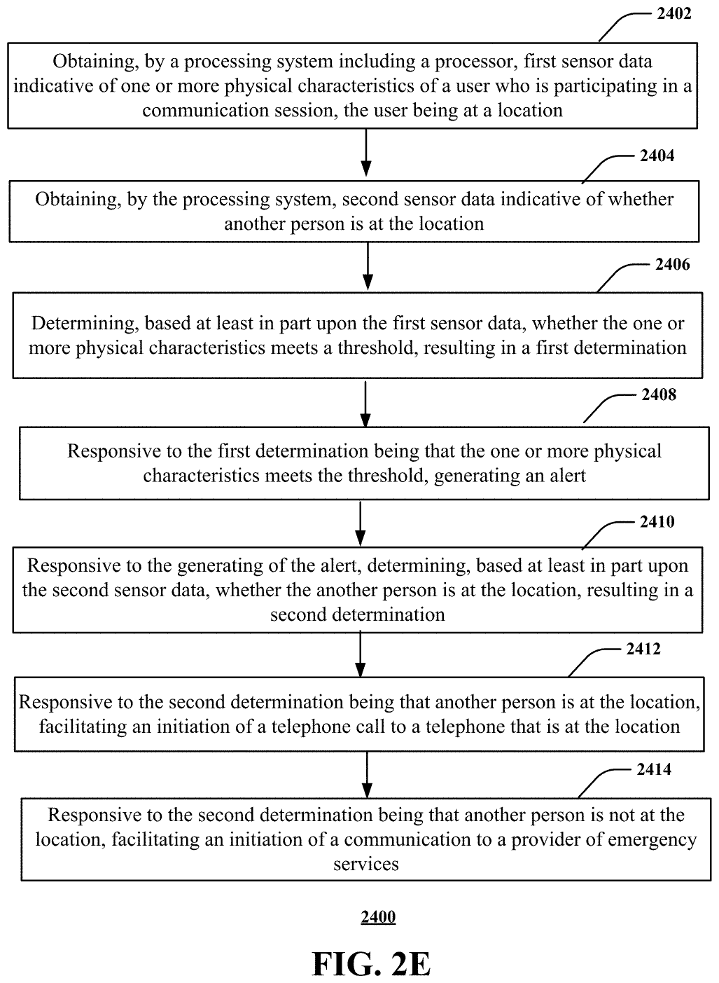

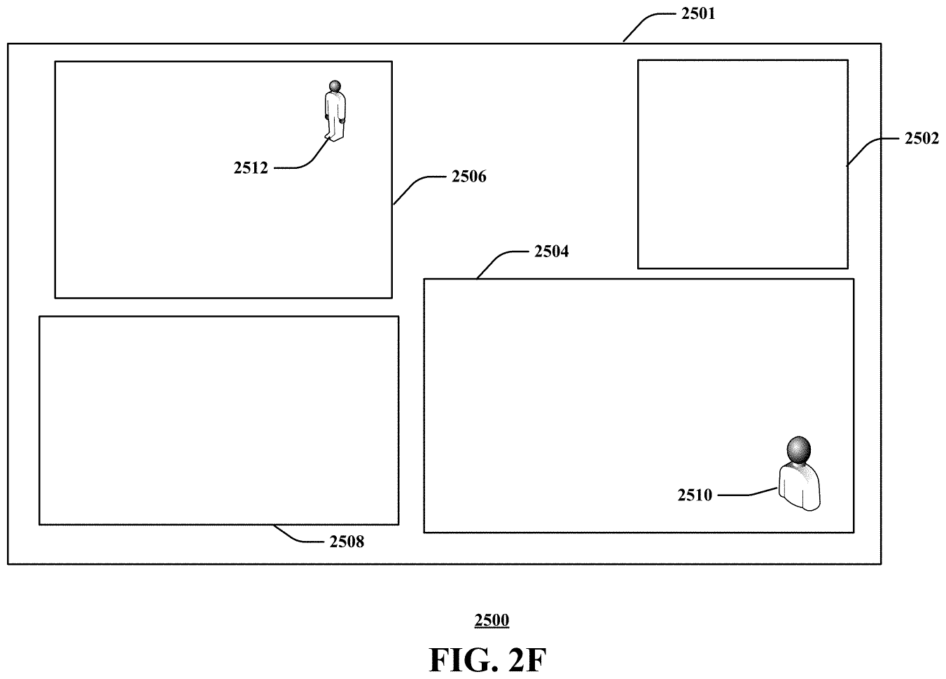

Referring now to FIG. 2A, this is a block diagram illustrating an example, non-limiting embodiment of a system 2000 (that can function fully or partially within the communication network of FIG. 1) in accordance with various aspects described herein. As seen in this figure, VA 2002 (which can comprise software, firmware, hardware, or a combination thereof) is provided. VA 2002 can tie-in (e.g., bidirectionally communicate with) security system 2004 (see arrow “A”). In various examples, security system 2004 can be a security system of a house, a security system of an apartment, a security system of an office, or any combination thereof. In various examples, the security system 2004 can detect the presence of an intruder 2006 (see arrow “B). Further, based on the tie-in between the VA 2002 and the security system 2004, the VA 2002 can facilitate actions such as described herein—e.g., to prevent the intruder 2006 from surprising the user (see arrow “C”). Further still, VA 2002 can monitor physical parameters (e.g., “vital stats”) of the user (see arrow “D”). In this example, the user can be communicating via virtual environment 2008. In another example, the user (whose physical parameters are being monitored) can be communicating via mixed/augmented environment 2010. In another example, virtual environment 2008 and mixed/augmented environment 2010 can be in bidirectional communication (see arrow “E”). Further still, VA 2002 can filter incoming calls/messages and/or communicate to specific contacts (see arrow “F”). In this example, the filtering/communicating is in connection with mixed/augmented environment 2010. In another example, the filtering/communicating can be in connection with virtual environment 2008. Further still, VA 2002 can alert emergency personnel 2012 if, for example, a user's “vital stats” indicate a health emergency (see arrow “G”).

Referring now to FIG. 2B, this is a block diagram illustrating an example, non-limiting embodiment of a system flow 2100 (that can be implemented fully or partially within the communication network of FIG. 1) in accordance with various aspects described herein. As seen in this figure, the system VA can perform (and/or facilitate) various functions. More particularly (see element 2102), the system VA can create a communication session (e.g., an XR communication session). Further (see element 2104), the system VA can perform internal & external monitoring. Further (see element 2106), the system VA can provide one or more alerts to the user when there are vital stat(s) degradation. Further (see element 2108), the system VA can provide one or more alerts to family/EMS depending on severity of health condition. Further (see element 2110), the system VA can “filter” general outreach, but “allow” message(s) from specified family members, and close contacts/emergency situations. Further (see element 2112), the system VA can tie-in with sensors and security system for perimeter—provides alerts for the user. See also, element 2112A—intruder entry ties-in with the security system to call EMS (and/or other personnel); and element 2112B—family member alerts are a notification indicating nearby presence. Further (see element 2114), the system VA can provide a map of the room and house/office building to show where external (other) people are located relative to the user/communication session (e.g., XR communication session).

Reference will now be made to a description of a system according to another embodiment. This embodiment can provide certain internal monitoring (see also, for example, element 2104 of FIG. 2B). More particularly, such internal monitoring can include:
1. Virtual Assistant (VA) allows entry (or re-entry) of a user into a virtual, augmented, and/or mixed environment.
   Upon entry: VA measures the specified biological health metrics (e.g., blood pressure, heart rate, oxygen %, blood sugar) via a clipped-on device, and/or syncs up with other device(s) such as watch/band/ring that is measuring these metrics.
   VA continues to monitor throughout the session.
2. VA has an internal health session interface that the user can access. It has the health stats for the session, and also previous session data that is presented in a graph format for comparison/trend identification.
   VA has an icon (e.g., heart shaped) that the user can click on throughout the session. The icon has a visual and audio indicator that prompts the user to check (e.g., the heart icon starts beating/pulsing, accompanied by a background sound indicating that there is a matter that needs to be observed (e.g., lower blood sugar, blood pressure)). The VA can also (or alternatively) alert the user post-session regarding negative trends (e.g., over the past few days, system has recorded lower oxygen levels compared to previous week/months).
3. VA alerts to body function degradation/health concerns and based on pre-authorized settings alerts family member/emergency contact and/or calls for emergency services, and flags/tags the program to ensure that the user is able to return to this spot where they left off.
   VA alerts user if the user is, for example, losing balance and/or falling—by monitoring the position of the user throughout the session.
   VA tracks if the user moves from standing to sitting (or vice versa), but also notes if the user trips/falls/loses balance.
   VA activates contacts/emergency services based on pre-determined settings.
   VA stops the program and allows the user to get back on track (e.g., waits for the user to pick up a fallen headset and does a quick body function check to ensure that there is no major health issue that needs to be alerted).
   VA records interruptions such as this, and logs patterns of behavior (e.g., multiple “falls”, or “falling asleep”—which may be early signs of poor physical/mental health quality).
   VA shares this data with the user, and if there is no positive change, VA can “force” user action by stating this information will be shared with family and/or health professional.

Reference will now be made to a description of a system according to another embodiment. This embodiment can provide certain internal monitoring (see also, for example, element 2104 of FIG. 2B). More particularly, such internal monitoring can include:
1. VA provides a pattern analysis to the user post-session—example: blood sugar drops after 3 hours of being within a session.
   For those using VR for exercise or health, VA provides a read-out of changes (improvements and/or declines) in health.
   VA provides analysis of patterns such as trending towards negative and suggests appropriate action depending on the severity.
   When there are positive patterns, VA tracks the path/activities that occurred so that the user can repeat successes and avoid activities that are not providing results.
   User can be “reminded” by the system to repeat positive behaviors (e.g., your blood sugar is low—in the past you've eaten something that took care of this issue).
2. VA monitors whether the user is losing accuracy by tracking eye patterns that indicate exhaustion/sleep—and is authorized to make executive decisions to switch off system.
   VA monitors whether the user has fallen asleep and switches off the system after noting where the program has “left off” so that the user can “get back to the spot” where they left off.
      VA offers options to come back to where the user was when the system was switched off, or when the body function alert was noted by VA.

Reference will now be made to a description of a system according to another embodiment. This embodiment can provide certain internal monitoring (see also, for example, element 2104 of FIG. 2B). More particularly, such internal monitoring can include:
1. In a matter of extreme health impact (e.g., signs of a heart attack or collapse), VA makes the decision to “call for help”—an alert through the communication session with other attendees, and/or a virtual “reach out” or phone call with a specified family member/close friend, or if necessary to local emergency services depending on the health condition.
   VA outgoing call can indicate through special identifier that this is the User's Emergency VA call so that the family member understands that this is a health/urgent call.
      Present a specific icon/text/audio alert for emergency contact within the communication session indicating a health emergency for a co-session attendee
      Identifier can be caller ID showing up as <User's name—Health Emergency>.
      VA can try second and third number if contacts are not reachable.
      VA can tie-in with emergency services and share “vital stats” of the user.
      VA can tie-in with Smart Home Security system to allow entry of emergency service personnel.
2. VA acts as a “filter” for what alerts come through the current communication session (e.g., XR communication session)
   Example: if a phone/video call comes in from family member/close friend/emergency contact that is attempting to reach the user, the VA will allow the session to be interrupted/placed “on hold” while user is put in contact.
      Depending on whether the user wants to allow interruptions or not (can be a predetermined setting)
         For example, based upon the predetermined settings, VA will allow all/some/no interruptions to the communication session.

Reference will now be made to a description of a system according to another embodiment. This embodiment can provide certain external monitoring (see also, for example, element 2104 of FIG. 2B). More particularly, such external monitoring can include:
1. VA monitors both external (e.g., physical) and internal (e.g., VR) functions while in a communication session, and alerts/ties external alerts and notifications with the session in experiences as needed.
   VA can tie-in with Smart Home, Connected Car, and/or other personal device(s) to “monitor” who is on premise with the user, and what is occurring externally while the user is in VR/AR session.
      User can track family member (or other person) via this tie-in process to ensure that they are on track/reaching within the specified time. Accordingly, if there are delays (e.g., traffic, road closure) the system can alert the user of the situation so that the user is aware of the latest status. This can be important because the user would typically need to continue to maintain awareness of the world outside the virtual/immersive environment.
   VA can “alert” the user regarding surroundings outside of the VR/AR space, including when people enter the same physical space where the user in a VR/AR session.
   The VA can have a sensor feedback system. When the sensors are placed at key entry points, doorway, and/or stairs, it alerts the user when those sensors are activated, indicating the presence of another person.
2. VA can provide varying levels of alerts to the user during VR/AR session so that the user is not caught “off guard” if someone enters the same room.
   VA can distinguish between family member and intruder, and the alerts are provided in that context.
   Alerts can include “perimeter alerts” which monitor a specified distance and/or location from which the user would receive an alert within the VR/AR system.
      This alert can be pre-determined within the user's profile, and can offer options including color-coded alert/accompanied by audio/text describing the alert.
      This alert can also activate a buzzer or other physical means of alerting the user.
      Depending on the pre-determined settings, VA can pause (or attempt to pause) the current session program, and provide options to the user to continue or exit the session (after tagging/flagging where the user left off).
         VA can provide (or attempt to provide) alternative arrangement for the user to continue the session in an alternative fashion.
         VA can (e.g., upon pre-determined settings/authorization) alert other(s) within the session that the user was interrupted due to a perimeter alert.
3. VA can provide a map of the house/perimeter, that can be accessed within the VR/AR session—that indicates where the intruder is relative to the VR/AR session.
   User receives the alert and is able to switch over to the “perimeter view” that indicates the position of the intruder.
   User can access camera/live footage, and to determine identity of the intruder.

Reference will now be made to a description of a system according to another embodiment. This embodiment can provide certain external monitoring (see also, for example, element 2104 of FIG. 2B). More particularly, such external monitoring can include:
1. VA can tie-in with Home Security system to prevent people from entering the home/premise.
   VA can determine through preset qualifiers for what is considered an “intrusion” (e.g., unidentified stranger is entering) vs an “alert” (e.g., family member(s are entering).
      VA can actively engage with home security systems to alert authorities and/or take specific action to address an intrusion.
      VA can actively engage with emergency personnel providing location and physical state (e.g., “vital stats”) of the user who is in a communication session and may not be aware/in a position to react to external happenings.

Referring now to FIG. 2C, various steps of a method 2200 according to an embodiment are shown. As seen in this FIG. 2C, step 2202 comprises monitoring a communication session in which a user participates, the user being at a location. Next, step 2204 comprises obtaining sensor information indicative of a physical environment in a vicinity of the location. Next, step 2206 comprises determining, based at least in part upon the sensor information, whether a person other than the user is in the physical environment, resulting in a determination. Next, step 2208 comprises responsive to the determination being that the person is in the physical environment: categorizing, based at least in part upon the sensor information, the person into one class of a plurality of classes of people; generating an alert, the alert indicating to which class the person has been categorized; and providing the alert via the communication session to a communication device being used by the user.

While for purposes of simplicity of explanation, the respective processes are shown and described as a series of blocks in FIG. 2C, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methods described herein.

Referring now to FIG. 2D, various steps of a method 2300 according to an embodiment are shown. As seen in this FIG. 2D, step 2302 comprises monitoring an extended reality (XR) communication session in which a user participates, the user being located in a room in a building. Next, step 2304 comprises communicating with one or more sensors that are disposed in the building, that are disposed on the building, or any combination thereof. Next, step 2306 comprises determining, based at least in part upon data from the one or more sensors, a location in the building of a person other than the user. Next, step 2308 comprises presenting on a communication device being used by the user, via the XR communication session, a map, the map including a first icon indicative of a first location of the user in the building and a second icon indicative of a second location of the person in the building.

While for purposes of simplicity of explanation, the respective processes are shown and described as a series of blocks in FIG. 2D, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methods described herein.

Referring now to FIG. 2E, various steps of a method 2400 according to an embodiment are shown. As seen in this FIG. 2E, step 2402 comprises obtaining, by a processing system including a processor, first sensor data indicative of one or more physical characteristics of a user who is participating in a communication session, the user being at a location. Next, step 2404 comprises obtaining, by the processing system, second sensor data indicative of whether another person is at the location. Next, step 2406 comprises determining, based at least in part upon the first sensor data, whether the one or more physical characteristics meets a threshold, resulting in a first determination. Next, step 2408 comprises responsive to the first determination being that the one or more physical characteristics meets the threshold, generating an alert. Next, step 2410 comprises responsive to the generating of the alert, determining, based at least in part upon the second sensor data, whether the another person is at the location, resulting in a second determination. Next, step 2412 comprises responsive to the second determination being that another person is at the location, facilitating an initiation of a telephone call to a telephone that is at the location. Next, step 2414 comprises responsive to the second determination being that another person is not at the location, facilitating an initiation of a communication to a provider of emergency services.

While for purposes of simplicity of explanation, the respective processes are shown and described as a series of blocks in FIG. 2E, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methods described herein.

Referring now to FIG. 2F, this is a block diagram illustrating an example, non-limiting embodiment of a map 2500 in accordance with various aspects described herein. In this example, the map 2500 shows a top-down view of four rooms 2502, 2504. 2506, 2508 of a house 2501. Further, in this example, a first icon 2510 is shown depicting where a user of a system is in the house and a second icon 2512 is shown depicting where another person is in the house. In one example, the first icon and the second icon are visually different.

As described herein, various embodiments can be applied in the context of a use case where a user in a communication session (e.g., an XR session) may otherwise get caught “off guard” when another person enters the same room as the user. In one example, a VA can distinguish between a family member and an intruder, and the alert(s) can be provided in that context. In another example, a VA can tie-in with a security system (e.g., a home security system and/or an office security system) and/or with sensor(s) that are placed at strategic locations so that the user is alerted when an intruder approaches. In another example, a VA can provide a visual map of where the “intruder” is, and/or provide a camera tie-in so that the user can see the intruder.

As described herein, various embodiments can be applied in the context of a use case where a user in a communication session (e.g., an XR session) may have health issues that they may not be aware of. In one example, a VA could monitor “vital stats” and present these to the user (and/or family member and/or emergency medical services) depending on the severity of the issue. In another example, a VA can intervene and report to the user when there is a negative trend of health and/or vital signs over a period of time.

As described herein, various embodiments can be applied in the context of a use case where a user in a communication session (e.g., an XR session) is experiencing a medical issue. In one example, a VA can alert family member(s) and/or emergency medical services. In another example, a VA can tie-in with hospital/ambulance and report on the user's location and “vital stats” so that medical personnel would be able to attend to the user upon arrival. In another example, a VA can tie-in to a security system so that medical technicians would be allowed entry into the home. In another example, a VA can record/track the sequence of events and provide such sequence of events to the doctor/hospital and/or family members so that they understand the circumstance.

As described herein, various embodiments can provide a VA (e.g., a single system) that monitors external (e.g., physical) and internal (e.g., VR/AR) functions while in a session, and alerts/ties external alerts and notifications within the session. In various examples, the VA can distinguish between intruders and family members (e.g., by syncing with home security system and system of sensors). In various examples, the VA can provide varying levels of alerts to the user during the communication session (e.g., XR session) so that the user is not caught “off guard” if someone enters the same room. In various examples, the VA can act as a “filter” for what alert(s) come through the current communication session (e.g., so that the user is not disturbed). In various examples, the VA can monitor standard (or pre-specified) vital body functions—such as heart rate, blood pressure, blood sugar, breathing pattern, oxygen level, etc. In various examples, the VA can alert to body function degradation and/or health concerns (the alerts can be based upon, for example, pre-authorized settings). In various examples, the alert(s) can comprise a call (and/or other communications) to one or more family members and/or to one or more emergency contacts and/or to emergency services. In various examples, the VA can flag/tag the communication session program to ensure that the user is able to return to this spot where they left off. In various examples, the VA can monitor whether the user is losing accuracy (e.g., by tracking eye patterns that indicate exhaustion/sleep) and the VA can be authorized to make executive decisions to switch off system (e.g., switch of the communication session). In various examples, the VA can monitor standard (or pre-specified) body functions—such as heart rate, blood pressure, blood sugar, breathing pattern, oxygen level, etc. and can note when certain patterns of these functions are being observed. This information regarding such observed patterns can be shared in a post-session with varying levels of alarm in proportion to the type/pattern of issues observed. In various examples, based upon such observed patterns, the VA can suggest (and/or implement) certain actions (e.g., required actions) to address the issue.

As described herein, various embodiments can provide for connected devices/cross-platform lifestyle management. For example, mechanisms can be provided to facilitate interaction from virtual to physical environment (e.g., related to how a user interacts with family members and society between XR/VR/AR/mixed environments). In another example, mechanisms can be provided for social/professional networking within XR/VR/AR/mixed environments (e.g., mechanisms can facilitate a virtual assistant/virtual companion that can enable interactions—both social and professional; mechanisms can facilitate a person setting up “filters” and/or “thresholds” for interacting with the general public, professional network, friends network and/or family).

As described herein, various embodiments can provide for a tie-in between health-wellness-family-communication. For example, conventional systems that are typically in place to monitor health can be tied-in with virtual healthcare—for instance, communication of such health/well-being to caregivers and family members who may not be able to be physically present).

As described herein, various embodiments can provide for identity, security, privacy, permissions, and/or thresholds. For example, mechanisms can be provided for management of user identity and security within mixed environments. In another example, mechanisms can be provided for controlling what a virtual assistant/virtual companion would have access to, and with whom/when a virtual assistant/virtual companion would share information.

As described herein, various embodiments can provide virtual assistants and/or virtual companions that bring together mixed realities within day-to-day life. In various examples, such virtual assistants and/or virtual companions can be implemented in the context of augmented reality (AR), virtual reality (VR), telecommunications, messaging applications, VoIP, connected cars; smart cars, robotics industry, healthcare industry, hospitals, insurance, ambulatory services, virtual healthcare services, security & smart home security, and/or gaming/technology.

As described herein, various embodiments can provide a virtual assistant (e.g., a software application) that monitors external (e.g., physical) and internal (e.g., VR) functions while in a communication session, and alerts/ties external alerts and notifications with the communication session in experiences as needed. In various examples, the virtual assistant ties-in with a Smart Home, a Connected Car, and/or other personal device(s) to “monitor” who is on premise with the user, and what is occurring externally while the user is in a communication session. In various examples, the virtual assistant “alerts” the user as to surroundings outside of the VR/AR space, including when people enter the same physical space where the user in a VR/AR session.

As described herein, various embodiments can provide a virtual assistant (e.g., a software application) that provides varying levels of alerts to the user during a communication session (e.g., a VR and/or AR session) so that the user is not caught “off guard” if someone enters the same room. In various examples, alerts can include “perimeter alerts” which monitor a specified distance and/or location from which the user would receive an alert within the VR/AR system. In various examples, an alert can be pre-determined within the user's profile, and can offer options including color-coded alert/accompanied by audio/text describing the alert.

As described herein, various embodiments can provide: (1) a VA that provides varying levels of alerts to the user during a communication session (e.g., a VR session and/or an AR session) so that the user is not caught “off guard” if someone enters the same room; (2) a VA that ties-in with a Home Security system to prevent people from entering the home/premise; (3) a VA that acts as a “filter” for what alerts come through the current communication session (e.g., a VR session and/or an AR session); (3) a VA that monitors standard (or pre-specified) body functions—such as heart rate, blood pressure, blood sugar, breathing pattern, oxygen level, etc.; (4) a VA that alerts to body function degradation/health concerns and, based on pre-authorized settings, alerts family member/emergency contact and/or calls for emergency services, and flags/tags the program to ensure that the user is able to return to this spot where they left off; (5) a VA that monitors whether the user is losing accuracy by tracking eye patterns that indicate exhaustion/sleep—and is authorized to make executive decisions to switch off system; and/or (6) a VA that monitors standard (or pre-specified) body functions—such as heart rate, blood pressure, blood sugar, breathing pattern, oxygen level, etc. and notes when certain patterns of these functions are being observed (this information can be shared by the VA with the user in a post-session with varying levels of alarm in proportion to the type/pattern of issues observed; further, the VA can suggest (and/or or implement) required actions to address the issue.

As described herein, various embodiments can provide a VA that ties-in with Home Security system to prevent people from entering the home/premise. In various examples: (a) the VA can distinguish between family member and intruder, and corresponding visual/audio indicators will reflect this; (b) the VA can determine through preset qualifiers for what is considered an “intrusion” (e.g., that a suspicious person is entering) vs an “alert” (e.g., that a family member is entering); (c) the VA can provide a visual map of where the “intruder” (or other person) is, and can provide a camera tie-in; and/or (d) the VA can actively engage with home security systems to alert authorities and/or take specific action to address an intrusion.

As described herein, various embodiments can provide a VA that acts as a “filter” for what alerts come through the communication session (e.g., a VR session and/or an AR session). In various examples: (a) if a phone/video call comes in from family member/close friend/emergency contact that is attempting to reach the user, the VA will allow the session to be interrupted/placed “on hold” while user is put in contact; (b) whether or not the VA permits the interruption can depend upon whether the user had indicated permission to allow interruptions or not (e.g., via one or more predetermined settings); and/or (c) depending upon one or more predetermined settings the VA will allow all/some/no interruptions to the communication session (e.g., a VR session and/or an AR session).

As described herein, various embodiments can provide a VA that monitors whether the user is losing accuracy by tracking eye patterns that indicate exhaustion/sleep—and is authorized to make executive decisions to switch off system. In various examples: (a) the VA can monitor whether the person has fallen asleep and switches off the system after noting where the program has “left off” so that the user can “get back to the spot” where they left off; and/or (b) the VA can offer options to come back to where the user was when the system was switched off, or when the body function alert was noted by the VA.

As described herein, various embodiments can provide a VA that monitors standard (or pre-specified) body functions—such as heart rate, blood pressure, blood sugar, breathing pattern, oxygen level, etc. and notes when certain patterns of these functions are being observed. In various examples: the VA attempts to (or does) address within the communication session (e.g., a VR session and/or an AR session) by alerting the user via voice/text/video that they need to address the issue—depending on the severity (in one specific example, if the system VA recognizes that the person is falling asleep—a gentle “notification” that it is time to switch off and pick up later can be provided; in another specific example, if the system VA recognizes that the user is having heart or blood pressure issues the VA can force a switch off of the communication session followed by the appropriate pre-determined emergency actions).

As described herein, various embodiments can provide a VA that provides a pattern analysis to the user in a post-session—example: let the user know that blood sugar drops after 3 hours of being within a session. In various examples: (a) for those using VR/AR for exercise or health, the VA can provide a read-out of changes (improvements and/or declines) in health; (b) the VA can provide analysis of patterns—for instance, that patterns are trending towards negative (in such a case, the VA can suggest appropriate action depending on the severity); and/or (c) when there are positive patterns, the VA can track the path/activities that occurred so that the user can repeat successes and avoid activities that are not providing positive results.

As described herein, various embodiments can provide tracking of events inside of a communication session (e.g., a VR session and/or an AR session) and/or outside of the communication session.

As described herein, various embodiments can provide for identifying: (a) a major health crisis; (b) a user falling asleep; and/or (c) a user losing balance.

As described herein, various embodiments can provide for interrupting a user (e.g., in the case of an urgent situation) or not interrupting the user (e.g., in the case of a non-urgent situation). In various examples, different levels of contacts can be associated with an interrupt or not interrupt permission.

As described herein, various embodiments can provide for tracking thirst (e.g., low electrolytes) and/or hunger of a user in a communication session (e.g., a VR session and/or an AR session). In various examples, a communication channel can be kept open to permit the user to be interrupted with food and/or beverage options (e.g., when it is detected that the user is hungry and/or thirsty). In various examples, a communication channel can be kept open to permit the user to be presented with advertising (e.g., based upon a user profile that had been set-up in a personal environment and/or agree to use in a public setting (e.g., credits for using the system)).

As described herein, various embodiments can provide for presenting a perimeter alert (e.g. “who” is trying to enter). In one example, if a “stranger” is trying to enter (and/or enters), then the communication session (e.g., a VR session and/or an AR session) can be automatically ended so that the user can address the issue (a bookmark or the like can be provided to enable the user to return where the communication session ended). In another example, if a “family member” is trying to enter (and/or enters), then a less drastic action (other than ending the communication session) can be implemented (e.g., alert the user that a person is coming into the house and/or room).

As described herein, various embodiments can provide for presenting a visual map (e.g., showing where an “intruder” is relative to the user; showing where a “family member” is relative to the user).

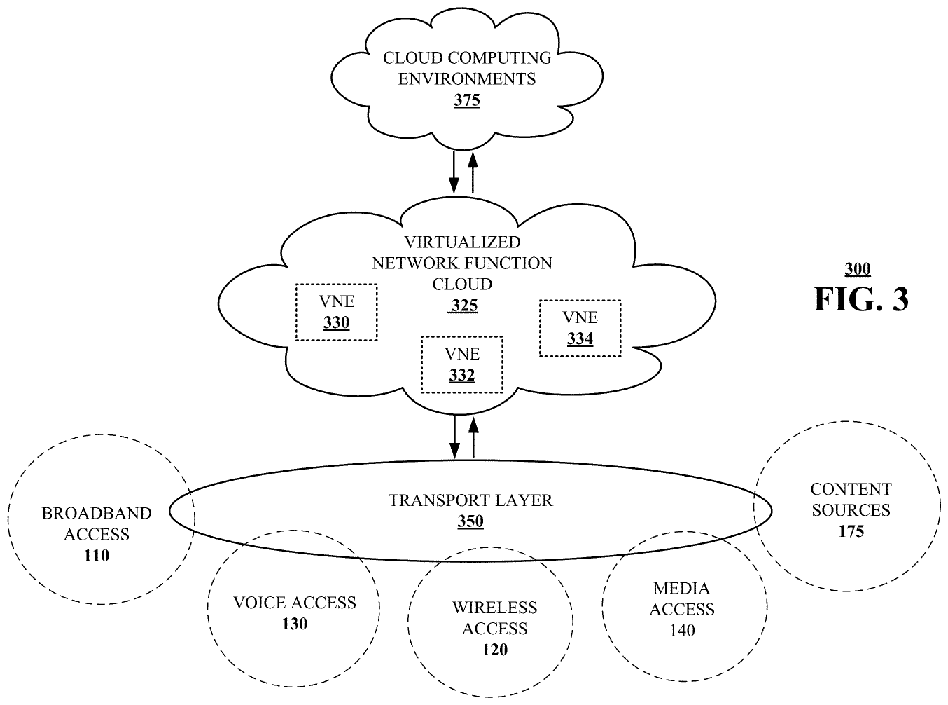

Referring now to FIG. 3, a block diagram 300 is shown illustrating an example, non-limiting embodiment of a virtualized communication network in accordance with various aspects described herein. In particular, a virtualized communication network is presented that can be used to implement some or all of the subsystems and functions of system 100, some or all of the subsystems and functions of system 2000, some or all of the functions of system flow 2100, and/or some or all of the functions of methods 2200, 2300, 2400. For example, virtualized communication network 300 can facilitate in whole or in part monitoring and/or responding during a user's communication session.

In particular, a cloud networking architecture is shown that leverages cloud technologies and supports rapid innovation and scalability via a transport layer 350, a virtualized network function cloud 325 and/or one or more cloud computing environments 375. In various embodiments, this cloud networking architecture is an open architecture that leverages application programming interfaces (APIs); reduces complexity from services and operations; supports more nimble business models; and rapidly and seamlessly scales to meet evolving customer requirements including traffic growth, diversity of traffic types, and diversity of performance and reliability expectations.

In contrast to traditional network elements—which are typically integrated to perform a single function, the virtualized communication network employs virtual network elements (VNEs) 330, 332, 334, etc. that perform some or all of the functions of network elements 150, 152, 154, 156, etc. For example, the network architecture can provide a substrate of networking capability, often called Network Function Virtualization Infrastructure (NFVI) or simply infrastructure that is capable of being directed with software and Software Defined Networking (SDN) protocols to perform a broad variety of network functions and services. This infrastructure can include several types of substrates. The most typical type of substrate being servers that support Network Function Virtualization (NFV), followed by packet forwarding capabilities based on generic computing resources, with specialized network technologies brought to bear when general purpose processors or general purpose integrated circuit devices offered by merchants (referred to herein as merchant silicon) are not appropriate. In this case, communication services can be implemented as cloud-centric workloads.

As an example, a traditional network element 150 (shown in FIG. 1), such as an edge router can be implemented via a VNE 330 composed of NFV software modules, merchant silicon, and associated controllers. The software can be written so that increasing workload consumes incremental resources from a common resource pool, and moreover so that it's elastic: so the resources are only consumed when needed. In a similar fashion, other network elements such as other routers, switches, edge caches, and middle-boxes are instantiated from the common resource pool. Such sharing of infrastructure across a broad set of uses makes planning and growing infrastructure easier to manage.

In an embodiment, the transport layer 350 includes fiber, cable, wired and/or wireless transport elements, network elements and interfaces to provide broadband access 110, wireless access 120, voice access 130, media access 140 and/or access to content sources 175 for distribution of content to any or all of the access technologies. In particular, in some cases a network element needs to be positioned at a specific place, and this allows for less sharing of common infrastructure. Other times, the network elements have specific physical layer adapters that cannot be abstracted or virtualized, and might require special DSP code and analog front-ends (AFEs) that do not lend themselves to implementation as VNEs 330, 332 or 334. These network elements can be included in transport layer 350.

The virtualized network function cloud 325 interfaces with the transport layer 350 to provide the VNEs 330, 332, 334, etc. to provide specific NFVs. In particular, the virtualized network function cloud 325 leverages cloud operations, applications, and architectures to support networking workloads. The virtualized network elements 330, 332 and 334 can employ network function software that provides either a one-for-one mapping of traditional network element function or alternately some combination of network functions designed for cloud computing. For example, VNEs 330, 332 and 334 can include route reflectors, domain name system (DNS) servers, and dynamic host configuration protocol (DHCP) servers, system architecture evolution (SAE) and/or mobility management entity (MME) gateways, broadband network gateways, IP edge routers for IP-VPN, Ethernet and other services, load balancers, distributers and other network elements. Because these elements don't typically need to forward large amounts of traffic, their workload can be distributed across a number of servers—each of which adds a portion of the capability, and overall which creates an elastic function with higher availability than its former monolithic version. These virtual network elements 330, 332, 334, etc. can be instantiated and managed using an orchestration approach similar to those used in cloud compute services.

The cloud computing environments 375 can interface with the virtualized network function cloud 325 via APIs that expose functional capabilities of the VNEs 330, 332, 334, etc. to provide the flexible and expanded capabilities to the virtualized network function cloud 325. In particular, network workloads may have applications distributed across the virtualized network function cloud 325 and cloud computing environment 375 and in the commercial cloud, or might simply orchestrate workloads supported entirely in NFV infrastructure from these third party locations.

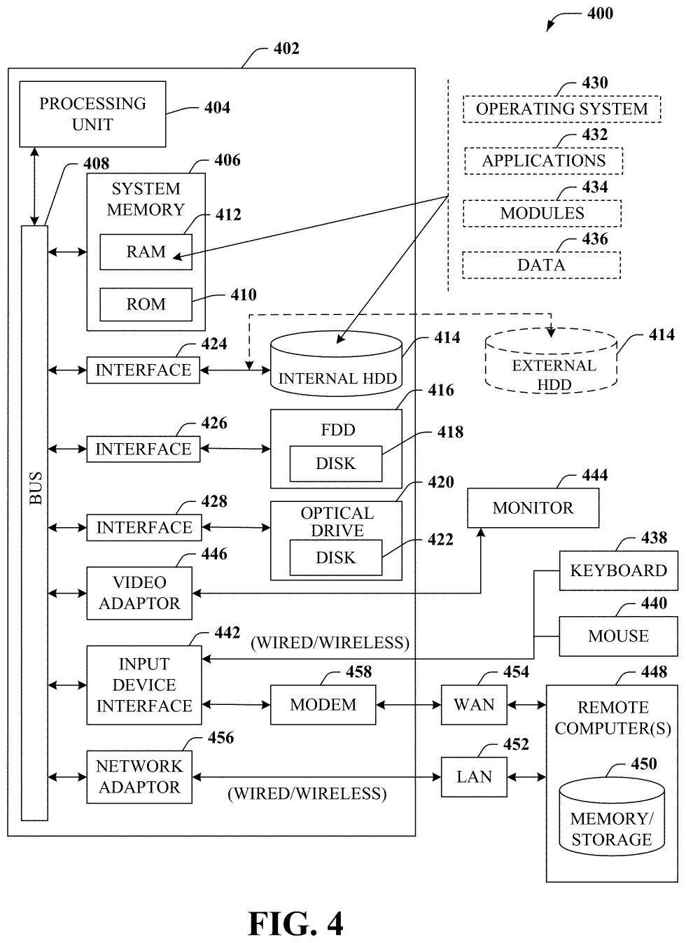

Turning now to FIG. 4, there is illustrated a block diagram of a computing environment in accordance with various aspects described herein. In order to provide additional context for various embodiments of the embodiments described herein, FIG. 4 and the following discussion are intended to provide a brief, general description of a suitable computing environment 400 in which the various embodiments of the subject disclosure can be implemented. In particular, computing environment 400 can be used in the implementation of network elements 150, 152, 154, 156, access terminal 112, base station or access point 122, switching device 132, media terminal 142, and/or VNEs 330, 332, 334, etc. Each of these devices can be implemented via computer-executable instructions that can run on one or more computers, and/or in combination with other program modules and/or as a combination of hardware and software. For example, computing environment 400 can facilitate in whole or in part monitoring and/or responding during a user's communication session.

Generally, program modules comprise routines, programs, components, data structures, etc., that perform particular tasks or implement particular abstract data types. Moreover, those skilled in the art will appreciate that the methods can be practiced with other computer system configurations, comprising single-processor or multiprocessor computer systems, minicomputers, mainframe computers, as well as personal computers, hand-held computing devices, microprocessor-based or programmable consumer electronics, and the like, each of which can be operatively coupled to one or more associated devices.

As used herein, a processing circuit includes one or more processors as well as other application specific circuits such as an application specific integrated circuit, digital logic circuit, state machine, programmable gate array or other circuit that processes input signals or data and that produces output signals or data in response thereto. It should be noted that while any functions and features described herein in association with the operation of a processor could likewise be performed by a processing circuit.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically comprise a variety of media, which can comprise computer-readable storage media and/or communications media, which two terms are used herein differently from one another as follows. Computer-readable storage media can be any available storage media that can be accessed by the computer and comprises both volatile and nonvolatile media, removable and non-removable media. By way of example, and not limitation, computer-readable storage media can be implemented in connection with any method or technology for storage of information such as computer-readable instructions, program modules, structured data or unstructured data.

Computer-readable storage media can comprise, but are not limited to, random access memory (RAM), read only memory (ROM), electrically erasable programmable read only memory (EEPROM), flash memory or other memory technology, compact disk read only memory (CD-ROM), digital versatile disk (DVD) or other optical disk storage, magnetic cassettes, magnetic tape, magnetic disk storage or other magnetic storage devices or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and comprises any information delivery or transport media. The term “modulated data signal” or signals refers to a signal that has one or more of its characteristics set or changed in such a manner as to encode information in one or more signals. By way of example, and not limitation, communication media comprise wired media, such as a wired network or direct-wired connection, and wireless media such as acoustic, RF, infrared and other wireless media.

With reference again to FIG. 4, the example environment can comprise a computer 402, the computer 402 comprising a processing unit 404, a system memory 406 and a system bus 408. The system bus 408 couples system components including, but not limited to, the system memory 406 to the processing unit 404. The processing unit 404 can be any of various commercially available processors. Dual microprocessors and other multiprocessor architectures can also be employed as the processing unit 404.

The system bus 408 can be any of several types of bus structure that can further interconnect to a memory bus (with or without a memory controller), a peripheral bus, and a local bus using any of a variety of commercially available bus architectures. The system memory 406 comprises ROM 410 and RAM 412. A basic input/output system (BIOS) can be stored in a non-volatile memory such as ROM, erasable programmable read only memory (EPROM), EEPROM, which BIOS contains the basic routines that help to transfer information between elements within the computer 402, such as during startup. The RAM 412 can also comprise a high-speed RAM such as static RAM for caching data.

The computer 402 further comprises an internal hard disk drive (HDD) 414 (e.g., EIDE, SATA), which internal HDD 414 can also be configured for external use in a suitable chassis (not shown), a magnetic floppy disk drive (FDD) 416, (e.g., to read from or write to a removable diskette 418) and an optical disk drive 420, (e.g., reading a CD-ROM disk 422 or, to read from or write to other high capacity optical media such as the DVD). The HDD 414, magnetic FDD 416 and optical disk drive 420 can be connected to the system bus 408 by a hard disk drive interface 424, a magnetic disk drive interface 426 and an optical drive interface 428, respectively. The hard disk drive interface 424 for external drive implementations comprises at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 402, the drives and storage media accommodate the storage of any data in a suitable digital format. Although the description of computer-readable storage media above refers to a hard disk drive (HDD), a removable magnetic diskette, and a removable optical media such as a CD or DVD, it should be appreciated by those skilled in the art that other types of storage media which are readable by a computer, such as zip drives, magnetic cassettes, flash memory cards, cartridges, and the like, can also be used in the example operating environment, and further, that any such storage media can contain computer-executable instructions for performing the methods described herein.

A number of program modules can be stored in the drives and RAM 412, comprising an operating system 430, one or more application programs 432, other program modules 434 and program data 436. All or portions of the operating system, applications, modules, and/or data can also be cached in the RAM 412. The systems and methods described herein can be implemented utilizing various commercially available operating systems or combinations of operating systems.

A user can enter commands and information into the computer 402 through one or more wired/wireless input devices, e.g., a keyboard 438 and a pointing device, such as a mouse 440. Other input devices (not shown) can comprise a microphone, an infrared (IR) remote control, a joystick, a game pad, a stylus pen, touch screen or the like. These and other input devices are often connected to the processing unit 404 through an input device interface 442 that can be coupled to the system bus 408, but can be connected by other interfaces, such as a parallel port, an IEEE 1394 serial port, a game port, a universal serial bus (USB) port, an IR interface, etc.

A monitor 444 or other type of display device can be also connected to the system bus 408 via an interface, such as a video adapter 446. It will also be appreciated that in alternative embodiments, a monitor 444 can also be any display device (e.g., another computer having a display, a smart phone, a tablet computer, etc.) for receiving display information associated with computer 402 via any communication means, including via the Internet and cloud-based networks. In addition to the monitor 444, a computer typically comprises other peripheral output devices (not shown), such as speakers, printers, etc.

The computer 402 can operate in a networked environment using logical connections via wired and/or wireless communications to one or more remote computers, such as a remote computer(s) 448. The remote computer(s) 448 can be a workstation, a server computer, a router, a personal computer, portable computer, microprocessor-based entertainment appliance, a peer device or other common network node, and typically comprises many or all of the elements described relative to the computer 402, although, for purposes of brevity, only a remote memory/storage device 450 is illustrated. The logical connections depicted comprise wired/wireless connectivity to a local area network (LAN) 452 and/or larger networks, e.g., a wide area network (WAN) 454. Such LAN and WAN networking environments are commonplace in offices and companies, and facilitate enterprise-wide computer networks, such as intranets, all of which can connect to a global communications network, e.g., the Internet.

When used in a LAN networking environment, the computer 402 can be connected to the LAN 452 through a wired and/or wireless communication network interface or adapter 456. The adapter 456 can facilitate wired or wireless communication to the LAN 452, which can also comprise a wireless AP disposed thereon for communicating with the adapter 456.

When used in a WAN networking environment, the computer 402 can comprise a modem 458 or can be connected to a communications server on the WAN 454 or has other means for establishing communications over the WAN 454, such as by way of the Internet. The modem 458, which can be internal or external and a wired or wireless device, can be connected to the system bus 408 via the input device interface 442. In a networked environment, program modules depicted relative to the computer 402 or portions thereof, can be stored in the remote memory/storage device 450. It will be appreciated that the network connections shown are example and other means of establishing a communications link between the computers can be used.

The computer 402 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This can comprise Wireless Fidelity (Wi-Fi) and BLUETOOTH® wireless technologies. Thus, the communication can be a predefined structure as with a conventional network or simply an ad hoc communication between at least two devices.

Wi-Fi can allow connection to the Internet from a couch at home, a bed in a hotel room or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE 802.11 (a, b, g, n, ac, ag, etc.) to provide secure, reliable, fast wireless connectivity. A Wi-Fi network can be used to connect computers to each other, to the Internet, and to wired networks (which can use IEEE 802.3 or Ethernet). Wi-Fi networks operate in the unlicensed 2.4 and 5 GHz radio bands for example or with products that contain both bands (dual band), so the networks can provide real-world performance similar to the basic 10BaseT wired Ethernet networks used in many offices.

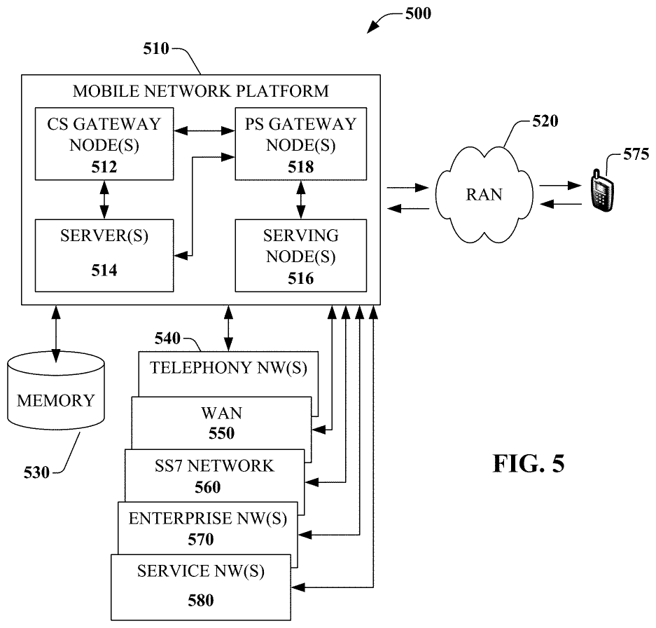

Turning now to FIG. 5, an embodiment 500 of a mobile network platform 510 is shown that is an example of network elements 150, 152, 154, 156, and/or VNEs 330, 332, 334, etc. For example, platform 510 can facilitate in whole or in part monitoring and/or responding during a user's communication session. In one or more embodiments, the mobile network platform 510 can generate and receive signals transmitted and received by base stations or access points such as base station or access point 122. Generally, mobile network platform 510 can comprise components, e.g., nodes, gateways, interfaces, servers, or disparate platforms, that facilitate both packet-switched (PS) (e.g., internet protocol (IP), frame relay, asynchronous transfer mode (ATM)) and circuit-switched (CS) traffic (e.g., voice and data), as well as control generation for networked wireless telecommunication. As a non-limiting example, mobile network platform 510 can be included in telecommunications carrier networks, and can be considered carrier-side components as discussed elsewhere herein. Mobile network platform 510 comprises CS gateway node(s) 512 which can interface CS traffic received from legacy networks like telephony network(s) 540 (e.g., public switched telephone network (PSTN), or public land mobile network (PLMN)) or a signaling system #7 (SS7) network 560. CS gateway node(s) 512 can authorize and authenticate traffic (e.g., voice) arising from such networks. Additionally, CS gateway node(s) 512 can access mobility, or roaming, data generated through SS7 network 560; for instance, mobility data stored in a visited location register (VLR), which can reside in memory 530. Moreover, CS gateway node(s) 512 interfaces CS-based traffic and signaling and PS gateway node(s) 518. As an example, in a 3GPP UMTS network, CS gateway node(s) 512 can be realized at least in part in gateway GPRS support node(s) (GGSN). It should be appreciated that functionality and specific operation of CS gateway node(s) 512, PS gateway node(s) 518, and serving node(s) 516, is provided and dictated by radio technology(ies) utilized by mobile network platform 510 for telecommunication over a radio access network 520 with other devices, such as a radiotelephone 575.

In addition to receiving and processing CS-switched traffic and signaling, PS gateway node(s) 518 can authorize and authenticate PS-based data sessions with served mobile devices. Data sessions can comprise traffic, or content(s), exchanged with networks external to the mobile network platform 510, like wide area network(s) (WANs) 550, enterprise network(s) 570, and service network(s) 580, which can be embodied in local area network(s) (LANs), can also be interfaced with mobile network platform 510 through PS gateway node(s) 518. It is to be noted that WANs 550 and enterprise network(s) 570 can embody, at least in part, a service network(s) like IP multimedia subsystem (IMS). Based on radio technology layer(s) available in technology resource(s) or radio access network 520, PS gateway node(s) 518 can generate packet data protocol contexts when a data session is established; other data structures that facilitate routing of packetized data also can be generated. To that end, in an aspect, PS gateway node(s) 518 can comprise a tunnel interface (e.g., tunnel termination gateway (TTG) in 3GPP UMTS network(s) (not shown)) which can facilitate packetized communication with disparate wireless network(s), such as Wi-Fi networks.

In embodiment 500, mobile network platform 510 also comprises serving node(s) 516 that, based upon available radio technology layer(s) within technology resource(s) in the radio access network 520, convey the various packetized flows of data streams received through PS gateway node(s) 518. It is to be noted that for technology resource(s) that rely primarily on CS communication, server node(s) can deliver traffic without reliance on PS gateway node(s) 518; for example, server node(s) can embody at least in part a mobile switching center. As an example, in a 3GPP UMTS network, serving node(s) 516 can be embodied in serving GPRS support node(s) (SGSN).

For radio technologies that exploit packetized communication, server(s) 514 in mobile network platform 510 can execute numerous applications that can generate multiple disparate packetized data streams or flows, and manage (e.g., schedule, queue, format . . . ) such flows. Such application(s) can comprise add-on features to standard services (for example, provisioning, billing, customer support . . . ) provided by mobile network platform 510. Data streams (e.g., content(s) that are part of a voice call or data session) can be conveyed to PS gateway node(s) 518 for authorization/authentication and initiation of a data session, and to serving node(s) 516 for communication thereafter. In addition to application server, server(s) 514 can comprise utility server(s), a utility server can comprise a provisioning server, an operations and maintenance server, a security server that can implement at least in part a certificate authority and firewalls as well as other security mechanisms, and the like. In an aspect, security server(s) secure communication served through mobile network platform 510 to ensure network's operation and data integrity in addition to authorization and authentication procedures that CS gateway node(s) 512 and PS gateway node(s) 518 can enact. Moreover, provisioning server(s) can provision services from external network(s) like networks operated by a disparate service provider; for instance, WAN 550 or Global Positioning System (GPS) network(s) (not shown). Provisioning server(s) can also provision coverage through networks associated to mobile network platform 510 (e.g., deployed and operated by the same service provider), such as the distributed antennas networks shown in FIG. 1(s) that enhance wireless service coverage by providing more network coverage.

It is to be noted that server(s) 514 can comprise one or more processors configured to confer at least in part the functionality of mobile network platform 510. To that end, the one or more processor can execute code instructions stored in memory 530, for example. It is should be appreciated that server(s) 514 can comprise a content manager, which operates in substantially the same manner as described hereinbefore.

In example embodiment 500, memory 530 can store information related to operation of mobile network platform 510. Other operational information can comprise provisioning information of mobile devices served through mobile network platform 510, subscriber databases; application intelligence, pricing schemes, e.g., promotional rates, flat-rate programs, couponing campaigns; technical specification(s) consistent with telecommunication protocols for operation of disparate radio, or wireless, technology layers; and so forth. Memory 530 can also store information from at least one of telephony network(s) 540, WAN 550, SS7 network 560, or enterprise network(s) 570. In an aspect, memory 530 can be, for example, accessed as part of a data store component or as a remotely connected memory store.

In order to provide a context for the various aspects of the disclosed subject matter, FIG. 5, and the following discussion, are intended to provide a brief, general description of a suitable environment in which the various aspects of the disclosed subject matter can be implemented. While the subject matter has been described above in the general context of computer-executable instructions of a computer program that runs on a computer and/or computers, those skilled in the art will recognize that the disclosed subject matter also can be implemented in combination with other program modules. Generally, program modules comprise routines, programs, components, data structures, etc. that perform particular tasks and/or implement particular abstract data types.

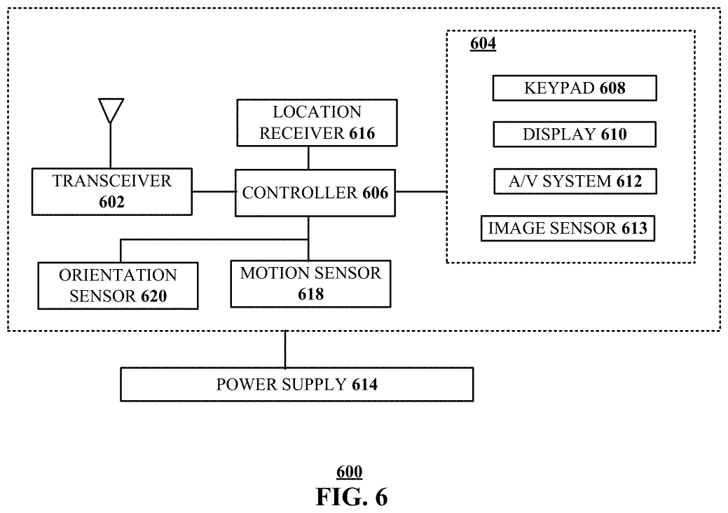

Turning now to FIG. 6, an illustrative embodiment of a communication device 600 is shown. The communication device 600 can serve as an illustrative embodiment of devices such as data terminals 114, mobile devices 124, vehicle 126, display devices 144 or other client devices for communication via either communications network 125. For example, computing device 600 can facilitate in whole or in part monitoring and/or responding during a user's communication session.

The communication device 600 can comprise a wireline and/or wireless transceiver 602 (herein transceiver 602), a user interface (UI) 604, a power supply 614, a location receiver 616, a motion sensor 618, an orientation sensor 620, and a controller 606 for managing operations thereof. The transceiver 602 can support short-range or long-range wireless access technologies such as Bluetooth®, ZigBee®, WiFi, DECT, or cellular communication technologies, just to mention a few (Bluetooth® and ZigBee® are trademarks registered by the Bluetooth® Special Interest Group and the ZigBee® Alliance, respectively). Cellular technologies can include, for example, CDMA-1×, UMTS/HSDPA, GSM/GPRS, TDMA/EDGE, EV/DO, WiMAX, SDR, LTE, as well as other next generation wireless communication technologies as they arise. The transceiver 602 can also be adapted to support circuit-switched wireline access technologies (such as PSTN), packet-switched wireline access technologies (such as TCP/IP, VoIP, etc.), and combinations thereof.

The UI 604 can include a depressible or touch-sensitive keypad 608 with a navigation mechanism such as a roller ball, a joystick, a mouse, or a navigation disk for manipulating operations of the communication device 600. The keypad 608 can be an integral part of a housing assembly of the communication device 600 or an independent device operably coupled thereto by a tethered wireline interface (such as a USB cable) or a wireless interface supporting for example Bluetooth®. The keypad 608 can represent a numeric keypad commonly used by phones, and/or a QWERTY keypad with alphanumeric keys. The UI 604 can further include a display 610 such as monochrome or color LCD (Liquid Crystal Display), OLED (Organic Light Emitting Diode) or other suitable display technology for conveying images to an end user of the communication device 600. In an embodiment where the display 610 is touch-sensitive, a portion or all of the keypad 608 can be presented by way of the display 610 with navigation features.

The display 610 can use touch screen technology to also serve as a user interface for detecting user input. As a touch screen display, the communication device 600 can be adapted to present a user interface having graphical user interface (GUI) elements that can be selected by a user with a touch of a finger. The display 610 can be equipped with capacitive, resistive or other forms of sensing technology to detect how much surface area of a user's finger has been placed on a portion of the touch screen display. This sensing information can be used to control the manipulation of the GUI elements or other functions of the user interface. The display 610 can be an integral part of the housing assembly of the communication device 600 or an independent device communicatively coupled thereto by a tethered wireline interface (such as a cable) or a wireless interface.

The UI 604 can also include an audio system 612 that utilizes audio technology for conveying low volume audio (such as audio heard in proximity of a human ear) and high volume audio (such as speakerphone for hands free operation). The audio system 612 can further include a microphone for receiving audible signals of an end user. The audio system 612 can also be used for voice recognition applications. The UI 604 can further include an image sensor 613 such as a charged coupled device (CCD) camera for capturing still or moving images.

The power supply 614 can utilize common power management technologies such as replaceable and rechargeable batteries, supply regulation technologies, and/or charging system technologies for supplying energy to the components of the communication device 600 to facilitate long-range or short-range portable communications. Alternatively, or in combination, the charging system can utilize external power sources such as DC power supplied over a physical interface such as a USB port or other suitable tethering technologies.

The location receiver 616 can utilize location technology such as a global positioning system (GPS) receiver capable of assisted GPS for identifying a location of the communication device 600 based on signals generated by a constellation of GPS satellites, which can be used for facilitating location services such as navigation. The motion sensor 618 can utilize motion sensing technology such as an accelerometer, a gyroscope, or other suitable motion sensing technology to detect motion of the communication device 600 in three-dimensional space. The orientation sensor 620 can utilize orientation sensing technology such as a magnetometer to detect the orientation of the communication device 600 (north, south, west, and east, as well as combined orientations in degrees, minutes, or other suitable orientation metrics).

The communication device 600 can use the transceiver 602 to also determine a proximity to a cellular, WiFi, Bluetooth®, or other wireless access points by sensing techniques such as utilizing a received signal strength indicator (RSSI) and/or signal time of arrival (TOA) or time of flight (TOF) measurements. The controller 606 can utilize computing technologies such as a microprocessor, a digital signal processor (DSP), programmable gate arrays, application specific integrated circuits, and/or a video processor with associated storage memory such as Flash, ROM, RAM, SRAM, DRAM or other storage technologies for executing computer instructions, controlling, and processing data supplied by the aforementioned components of the communication device 600.

Other components not shown in FIG. 6 can be used in one or more embodiments of the subject disclosure. For instance, the communication device 600 can include a slot for adding or removing an identity module such as a Subscriber Identity Module (SIM) card or Universal Integrated Circuit Card (UICC). SIM or UICC cards can be used for identifying subscriber services, executing programs, storing subscriber data, and so on.

The terms “first,” “second,” “third,” and so forth, as used in the claims, unless otherwise clear by context, is for clarity only and doesn't otherwise indicate or imply any order in time. For instance, “a first determination,” “a second determination,” and “a third determination,” does not indicate or imply that the first determination is to be made before the second determination, or vice versa, etc.

In the subject specification, terms such as “store,” “storage,” “data store,” data storage,” “database,” and substantially any other information storage component relevant to operation and functionality of a component, refer to “memory components,” or entities embodied in a “memory” or components comprising the memory. It will be appreciated that the memory components described herein can be either volatile memory or nonvolatile memory, or can comprise both volatile and nonvolatile memory, by way of illustration, and not limitation, volatile memory, non-volatile memory, disk storage, and memory storage. Further, nonvolatile memory can be included in read only memory (ROM), programmable ROM (PROM), electrically programmable ROM (EPROM), electrically erasable ROM (EEPROM), or flash memory. Volatile memory can comprise random access memory (RAM), which acts as external cache memory. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM). Additionally, the disclosed memory components of systems or methods herein are intended to comprise, without being limited to comprising, these and any other suitable types of memory.

Moreover, it will be noted that the disclosed subject matter can be practiced with other computer system configurations, comprising single-processor or multiprocessor computer systems, mini-computing devices, mainframe computers, as well as personal computers, hand-held computing devices (e.g., PDA, phone, smartphone, watch, tablet computers, netbook computers, etc.), microprocessor-based or programmable consumer or industrial electronics, and the like. The illustrated aspects can also be practiced in distributed computing environments where tasks are performed by remote processing devices that are linked through a communications network; however, some if not all aspects of the subject disclosure can be practiced on stand-alone computers. In a distributed computing environment, program modules can be located in both local and remote memory storage devices.

In one or more embodiments, information regarding use of services can be generated including services being accessed, media consumption history, user preferences, and so forth. This information can be obtained by various methods including user input, detecting types of communications (e.g., video content vs. audio content), analysis of content streams, sampling, and so forth. The generating, obtaining and/or monitoring of this information can be responsive to an authorization provided by the user. In one or more embodiments, an analysis of data can be subject to authorization from user(s) associated with the data, such as an opt-in, an opt-out, acknowledgement requirements, notifications, selective authorization based on types of data, and so forth.

Some of the embodiments described herein can also employ artificial intelligence (AI) to facilitate automating one or more features described herein. The embodiments (e.g., in connection with automatically monitoring and/or responding during a user's communication session) can employ various AI-based schemes for carrying out various embodiments thereof. Moreover, the classifier can be employed to determine a ranking or priority of each user, each communication session, each external event, and/or each external person. A classifier is a function that maps an input attribute vector, x=(x1, x2, x3, x4, . . . , xn), to a confidence that the input belongs to a class, that is, f(x)=confidence (class). Such classification can employ a probabilistic and/or statistical-based analysis (e.g., factoring into the analysis utilities and costs) to determine or infer an action that a user desires to be automatically performed. A support vector machine (SVM) is an example of a classifier that can be employed. The SVM operates by finding a hypersurface in the space of possible inputs, which the hypersurface attempts to split the triggering criteria from the non-triggering events. Intuitively, this makes the classification correct for testing data that is near, but not identical to training data. Other directed and undirected model classification approaches comprise, e.g., naïve Bayes, Bayesian networks, decision trees, neural networks, fuzzy logic models, and probabilistic classification models providing different patterns of independence can be employed. Classification as used herein also is inclusive of statistical regression that is utilized to develop models of priority.

As will be readily appreciated, one or more of the embodiments can employ classifiers that are explicitly trained (e.g., via a generic training data) as well as implicitly trained (e.g., via observing UE behavior, operator preferences, historical information, receiving extrinsic information). For example, SVMs can be configured via a learning or training phase within a classifier constructor and feature selection module. Thus, the classifier(s) can be used to automatically learn and perform a number of functions, including but not limited to determining according to predetermined criteria functions for monitoring and/or responding during a user's communication session, etc.

As used in some contexts in this application, in some embodiments, the terms “component,” “system” and the like are intended to refer to, or comprise, a computer-related entity or an entity related to an operational apparatus with one or more specific functionalities, wherein the entity can be either hardware, a combination of hardware and software, software, or software in execution. As an example, a component may be, but is not limited to being, a process running on a processor, a processor, an object, an executable, a thread of execution, computer-executable instructions, a program, and/or a computer. By way of illustration and not limitation, both an application running on a server and the server can be a component. One or more components may reside within a process and/or thread of execution and a component may be localized on one computer and/or distributed between two or more computers. In addition, these components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor, wherein the processor can be internal or external to the apparatus and executes at least a part of the software or firmware application. As yet another example, a component can be an apparatus that provides specific functionality through electronic components without mechanical parts, the electronic components can comprise a processor therein to execute software or firmware that confers at least in part the functionality of the electronic components. While various components have been illustrated as separate components, it will be appreciated that multiple components can be implemented as a single component, or a single component can be implemented as multiple components, without departing from example embodiments.

Further, the various embodiments can be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable device or computer-readable storage/communications media. For example, computer readable storage media can include, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

In addition, the words “example” and “exemplary” are used herein to mean serving as an instance or illustration. Any embodiment or design described herein as “example” or “exemplary” is not necessarily to be construed as preferred or advantageous over other embodiments or designs. Rather, use of the word example or exemplary is intended to present concepts in a concrete fashion. As used in this application, the term “or” is intended to mean an inclusive “or” rather than an exclusive “or”. That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Moreover, terms such as “user equipment,” “mobile station,” “mobile,” subscriber station,” “access terminal,” “terminal,” “handset,” “mobile device” (and/or terms representing similar terminology) can refer to a wireless device utilized by a subscriber or user of a wireless communication service to receive or convey data, control, voice, video, sound, gaming or substantially any data-stream or signaling-stream. The foregoing terms are utilized interchangeably herein and with reference to the related drawings.

Furthermore, the terms “user,” “subscriber,” “customer,” “consumer” and the like are employed interchangeably throughout, unless context warrants particular distinctions among the terms. It should be appreciated that such terms can refer to human entities or automated components supported through artificial intelligence (e.g., a capacity to make inference based, at least, on complex mathematical formalisms), which can provide simulated vision, sound recognition and so forth.

As employed herein, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components or any combination thereof designed to perform the functions described herein. Processors can exploit nano-scale architectures such as, but not limited to, molecular and quantum-dot based transistors, switches and gates, in order to optimize space usage or enhance performance of user equipment. A processor can also be implemented as a combination of computing processing units.

As used herein, terms such as “data storage,” data storage,” “database,” and substantially any other information storage component relevant to operation and functionality of a component, refer to “memory components,” or entities embodied in a “memory” or components comprising the memory. It will be appreciated that the memory components or computer-readable storage media, described herein can be either volatile memory or nonvolatile memory or can include both volatile and nonvolatile memory.

What has been described above includes mere examples of various embodiments. It is, of course, not possible to describe every conceivable combination of components or methodologies for purposes of describing these examples, but one of ordinary skill in the art can recognize that many further combinations and permutations of the present embodiments are possible. Accordingly, the embodiments disclosed and/or claimed herein are intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

In addition, a flow diagram may include a “start” and/or “continue” indication. The “start” and “continue” indications reflect that the steps presented can optionally be incorporated in or otherwise used in conjunction with other routines. In this context, “start” indicates the beginning of the first step presented and may be preceded by other activities not specifically shown. Further, the “continue” indication reflects that the steps presented may be performed multiple times and/or may be succeeded by other activities not specifically shown. Further, while a flow diagram indicates a particular ordering of steps, other orderings are likewise possible provided that the principles of causality are maintained.

As may also be used herein, the term(s) “operably coupled to”, “coupled to”, and/or “coupling” includes direct coupling between items and/or indirect coupling between items via one or more intervening items. Such items and intervening items include, but are not limited to, junctions, communication paths, components, circuit elements, circuits, functional blocks, and/or devices. As an example of indirect coupling, a signal conveyed from a first item to a second item may be modified by one or more intervening items by modifying the form, nature or format of information in a signal, while one or more elements of the information in the signal are nevertheless conveyed in a manner than can be recognized by the second item. In a further example of indirect coupling, an action in a first item can cause a reaction on the second item, as a result of actions and/or reactions in one or more intervening items.

Although specific embodiments have been illustrated and described herein, it should be appreciated that any arrangement which achieves the same or similar purpose may be substituted for the embodiments described or shown by the subject disclosure. The subject disclosure is intended to cover any and all adaptations or variations of various embodiments. Combinations of the above embodiments, and other embodiments not specifically described herein, can be used in the subject disclosure. For instance, one or more features from one or more embodiments can be combined with one or more features of one or more other embodiments. In one or more embodiments, features that are positively recited can also be negatively recited and excluded from the embodiment with or without replacement by another structural and/or functional feature. The steps or functions described with respect to the embodiments of the subject disclosure can be performed in any order. The steps or functions described with respect to the embodiments of the subject disclosure can be performed alone or in combination with other steps or functions of the subject disclosure, as well as from other embodiments or from other steps that have not been described in the subject disclosure. Further, more than or less than all of the features described with respect to an embodiment can also be utilized.

What is claimed is:

1. A device comprising:
a processing system including a processor; and
a memory that stores executable instructions that, when executed by the processing system, facilitate performance of operations, the operations comprising:
monitoring a communication session in which a user participates, the user being at a location;
obtaining sensor information indicative of a physical environment in a vicinity of the location;
determining, based at least in part upon the sensor information, whether a person other than the user is in the physical environment, resulting in a determination; and
responsive to the determination being that the person is in the physical environment:
categorizing, based at least in part upon the sensor information, the person into one class of a plurality of classes of people;
generating an alert, the alert indicating to which class the person has been categorized; and
providing the alert via the communication session to a communication device being used by the user.

2. The device of claim 1, wherein the communication session is an extended reality (XR) communication session comprising a virtual reality (VR) communication session, an augmented reality (AR) communication session, a mixed reality (MR) communication session, or any combination thereof.

3. The device of claim 1, wherein the location comprises a home of the user, an apartment of the user, an office of the user, or any combination thereof.

4. The device of claim 1, wherein the vicinity of the location comprises being in a home of the user, being on a property outside the home of the user; being in an apartment of the user, being in an office of the user, or any combination thereof.

5. The device of claim 1, wherein the sensor information is obtained from a visible light camera, an infrared light camera, a heat camera, a microphone, or any combination thereof.

6. The device of claim 5, wherein the determination being that the person is in the physical environment is made based upon visible light imaging data from the visible light camera, infrared light imaging data from the infrared light camera, heat imaging data from the heat camera, sound data from the microphone, or any combination thereof.

7. The device of claim 6, wherein the categorizing is performed via analysis of the visible light imaging data, the infrared light imaging data, the heat imaging data, the sound data, or any combination thereof.

8. The device of claim 1, wherein the plurality of classes of people comprise a family class and a non-family class, the family class including people who are related to the user and the non-family class excluding people who are related to the user.

9. The device of claim 8, wherein the people who are related to the user are immediate family members of the user.

10. The device of claim 9, wherein the non-family class comprises a delivery person subclass, an unidentified person subclass and a suspicious person subclass.

11. The device of claim 10, wherein the alert that is provided to the user via the communication session differs depending upon into which class or subclass the person has been categorized.

12. The device of claim 11, wherein:
in a first case that the person has been categorized into the family class, the alert is less intrusive;
in a second case that the person has been categorized into the non-family class, the alert is more intrusive.

13. The device of claim 11, wherein:
in a first case that the person has been categorized into the family class, the alert has a first level of intrusiveness;
in a second case that the person has been categorized into the unidentified person subclass, the alert has a second level of intrusiveness;
in a third case that the person has been categorized into the suspicious person subclass, the alert has a third level of intrusiveness;
the first level of intrusiveness being a lowest level of intrusiveness;
the second level of intrusiveness being between the first level of intrusiveness and the third level of intrusiveness; and
the third level of intrusiveness being a highest level of intrusiveness.

14. The device of claim 1, wherein:
the communication session comprises an extended reality (XR) communication session;
the providing the alert via the communication session to the communication device being used by the user comprises providing the alert by a visual indicator displayed a screen of the communication device being used by the user, providing the alert by an audio indicator produced by a speaker of the communication device being used by the user, or any combination thereof; and
the communication device being used by the user comprises a desktop computer, a laptop computer, a notebook computer, a tablet computer, a smartphone, head mounted glasses, head mounted goggles, or any combination thereof.

15. The device of claim 1, wherein:
the communication session comprises an extended reality (XR) communication session in which the user participates; and
the XR communication session in which the user participates is also participated in by one or more other users, each of the one or more other users being located at a respective other location.

16. A non-transitory machine-readable medium comprising executable instructions that, when executed by a processing system including a processor, facilitate performance of operations, the operations comprising:
monitoring an extended reality (XR) communication session in which a user participates, the user being located in a room in a building;
communicating with one or more sensors that are disposed in the building, that are disposed on the building, or any combination thereof;
determining, based at least in part upon data from the one or more sensors, a location in the building of a person other than the user; and
presenting on a communication device being used by the user, via the XR communication session, a map, the map including a first icon indicative of a first location of the user in the building and a second icon indicative of a second location of the person in the building.

17. The non-transitory machine-readable medium of claim 16, wherein the communicating comprises:
requesting, from the one or more sensors, the data; and
receiving, from the one or more sensors, the data.

18. The non-transitory machine-readable medium of claim 16, wherein:
the presenting on the communication device being used by the user the map comprises displaying the map on a display screen of the communication device being used by the user;
the first icon is visually different from the second icon; and
the XR communication session comprises a virtual reality (VR) communication session, an augmented reality (AR) communication session, a mixed reality (MR) communication session, or any combination thereof.

19. A method comprising:
obtaining, by a processing system including a processor, first sensor data indicative of one or more physical characteristics of a user who is participating in a communication session, the user being at a location;
obtaining, by the processing system, second sensor data indicative of whether another person is at the location;
determining, based at least in part upon the first sensor data, whether the one or more physical characteristics meets a threshold, resulting in a first determination;
responsive to the first determination being that the one or more physical characteristics meets the threshold, generating an alert;
responsive to the generating of the alert, determining, based at least in part upon the second sensor data, whether the another person is at the location, resulting in a second determination;
responsive to the second determination being that another person is at the location, facilitating an initiation of a telephone call to a telephone that is at the location; and
responsive to the second determination being that another person is not at the location, facilitating an initiation of a communication to a provider of emergency services.

20. The method of claim 19, wherein:
the one or more physical characteristics of the user comprise heart rate, blood pressure, blood sugar, breathing pattern, oxygen level, electrolyte level, eye movement, or any combination thereof;
the meeting of the threshold for the heart rate comprises the heart rate being too high or the heart rate being too low;
the meeting of the threshold for the blood pressure comprises the blood pressure being too high or the blood pressure being too low;
the meeting of the threshold for the blood sugar comprises the blood sugar being too high or the blood sugar being too low;
the meeting of the threshold for the breathing pattern comprises the breathing pattern being too fast or the breathing pattern being too slow;
the meeting of the threshold for the oxygen level comprises the oxygen level being too high or the oxygen level being too low;
the meeting of the threshold for the electrolyte level comprises the electrolyte level being too high or the electrolyte level being too low;
the meeting of the threshold for the eye movement comprises the eye movement being too fast or the eye movement being too slow;
the location is a house associated with the user, an office building associated with the user, or any combination thereof;
the second determination is based at least in part upon the another person being in the house, the another person being in the office building, or any combination thereof;
the telephone call is to a landline telephone, the landline telephone comprising a POTS telephone connection, a voice over IP (VOIP) telephone connection, or any combination thereof; and
the provider of emergency services comprises a doctor's office, a hospital, an ambulance service, a fire department, a police department, or any combination thereof.
